(19) United States
(12) Patent Application Publication
(10) Pub. No.: US 2026/0083464 A1
(43) Pub. Date: Mar. 26, 2026

(54) Osteotomy Guide

(71) Applicant: DrMarkInnovations.Vet LLC, Victoria, TX (US)

(72) Inventor: Mark Besancon, Victoria, TX (US)

(21) Appl. No.: 18/891,321

(22) Filed: Sep. 20, 2024

(51) Int. Cl. A61B 17/17

(52) U.S. Cl. CPC A61B 17/1732

(57) ABSTRACT

An osteotomy guide to improve accuracy and efficiency of tibial plateau leveling osteotomy (TPLO) procedure. The guide comprises a concave shaped body with two ends on the opposite sides of the body wherein both ends are flushed, or one end is flushed and the other end is extended to form a flare. The flare also comprises a first end which may be extended from the first end of the guide. The guide also comprises a tibial plateau facing inner surface and an outer surface opposite the inner surface. The inner surface has a notch or groove or notches or grooves, which can be used as a landmark for tibial rotation. Further, the area enclosed by the two ends and inner and outer surfaces has plurality of orifices or fixation holes that are used for placing, securing and inserting mechanisms to secure the guide. The guide is durable, reusable or single-use and available in different sizes.

### CROSS-REFERENCE TO RELATED APPLICATIONS

Non-Applicable

### FEDERALLY SPONSORED RESEARCH OR DEVELOPMENT

Non-Applicable

### FIELD OF THE INVENTION

The present invention, in general, relates to a guide that can be used during osteotomy procedures in animals. Specifically, this osteotomy guide is designed to improve the efficiency and accuracy of the tibial plateau leveling osteotomy (TPLO) procedure by addressing several issues including “blade drifting” (i.e., blade movement from its intended position during the TPLO procedure). This concave-shaped guide comprises a first end, a second end, an inner tibial plateau facing surface exhibiting a groove that functions as a landmark for rotation measurement of the tibia, and an outer surface that is opposite to the inner surface. The first end and second end of the guide may be flushed, or the second end may be flushed, and the body may have a flare protruding outward from the first end. The guide may have plurality of orifices in the area enclosed by the first end, second end, inner surface and outer surface of the guide. These orifices enable placement mechanisms to secure the guide during the procedure and include but are not limited to sta-pin, k-wire, or point to point clamp placement. The guide comes in various sizes that match the TPLO crescent blades, which vary based on the size of the animal's limb and involved anatomical features, and can be used for the left and right legs of the animals.

### BACKGROUND

One of the most common surgeries performed on animals, such as dogs, is the TPLO procedure. The animals who undergo this procedure generally have sustained injuries to their knees due to tearing of the cranial cruciate ligament (CCL), which is similar to anterior cruciate ligament (ACL) tear in humans. Animals with CCL tear cannot walk normally and experience pain when they try to do so because their shin bone or tibia slides forward with respect to their thigh bone or femur. If this is not addressed, it may cause further damage to the cartilage and bones and may lead to osteoarthritis.

The TPLO procedure itself was developed by Dr. Barclay Slocum over twenty years ago to address the CCL tear in animals by changing the angle and the position between the femur and the tibia. More specifically, the goal of the surgery is to reduce the shift of the tibia as the animal walks. The surgery comprises making a cut that is semicircular through the top of the tibia and rotating the top of the tibia to realign its position with respect to the femur and inserting a metal plate to secure the tibia. This provides stability to the animal when it walks and reduces inflammation and the likelihood of osteoarthritis.

Although the TPLO procedure has been practiced for a number of years, one of the common problems that veterinary surgeons face is “drifting” of the blade while making the cut through the top of the tibia. This leads to a cut that is not positioned exactly where the surgeon prefers, and which is detrimental to the overall functioning of the inserted device.

Hence, there is a need for a safe and efficient tool to increase the accuracy of the positioning of the cut made by the surgeon during the TPLO procedure and thus, enabling proper alignment of the tibia in relation to the femur of the animal.

While the inventor has set forth the best mode or modes contemplated in carrying out the present invention known to the inventor, such as to enable a person skilled in the art to make and practice the present invention, the preferred embodiments are, however, not intended to be limiting, but are, on the contrary, included in a non-limiting sense apt to amendments, alterations, alternatives and modifications, based primarily on the animal size, in light of specification and appended claims forming the current disclosure.

### BRIEF SUMMARY OF THE INVENTION

An osteotomy guide to help veterinary surgeons make an incision in the proximal tibial region during a TPLO procedure by eliminating blade “drifting” which results in improper device placement. This guide comprises a concave shaped body with a first end, second end, a tibial plateau facing inner surface and an outer surface that is opposite to the inner surface. The inner surface exhibits a groove or a notch that is used by the surgeon as a landmark to mark the point or position for rotation of the tibia. In the area enclosed by the first end, second end, inner surface and outer surface, are a plurality of orifices providing a channel therethrough the thickness of the present invention. These orifices can be laid out in different patterns, for example, they could be in a single line or multiple lines, occurring regularly or irregularly, of different shapes and sizes, or a combination thereof. The orifices are used to insert or place mechanisms to secure the guide that is the present invention to the anatomical features of an animal's leg. These mechanisms include but are not limited to pins (e.g., sta-fix pins), screws, rivets, k-wire, point to point clamps or a combination thereof. The guide is made of reusable and durable material including but not limited to stainless steel, resin, titanium, tungsten, carbon steel, a combination thereof, or any material suitable for use in surgery, such as osteotomy. The guide is available in different sizes that are compatible with TPLO crescent blades and the structure and size of the tibia of the animal undergoing the osteotomy.

Also described herein is the method of performing TPLO procedures on an animal using the guide that is the present invention. After sedating the animal, shaving the hair off the skin area where the procedure will be performed, an incision is made on the skin and forceps or fingers are used to expose the bone and muscle underneath the skin. The patellar tendon of the animal is protected, and the guide is placed on the surface of the proximal tibia by utilizing the “D” measurements, i.e. by measuring the absolute displacement of the tip of the osteotomy blade, for correct placement of the guide. The placement of the exhibited notch or groove is confirmed before securing the guide to the surface of the tibia using the securing mechanisms mentioned above. A curved incision along the inner surface of the guide is made using the osteotomy blade and the site for rotation of the tibia is marked in relation to the notch or groove on the guide. The tibia is then rotated so that the tibia and femur are aligned properly, and the rotated tibia is secured by the securing mechanisms mentioned above. The guide is then removed, and a metal bone plate is placed to set the tibia in desired position and then secured.

In a preferred embodiment of the present invention is an osteotomy guide. This guide comprises a body with tibial plateau facing inner surface, said inner surface along which an incision is made during osteotomy; and an outer surface that is opposite to the inner surface, wherein said body has a first end and a second end. This osteotomy guide prevents/eliminates blade “drift”, untoward movement and improves efficiency and accuracy of osteotomy procedures.

In one embodiment of the preferred osteotomy guide, the first end and second end of the body are flush or where the second end of the body is flush and said body has a flare protruding outward from the first end, said flare comprising a first end and a second end.

In yet another embodiment of the preferred osteotomy guide, the first end of the flare is an extension of the first end of the body and the second end of the flare is close to the patellar tendon of an animal undergoing an osteotomy procedure. Although the invention describes the flare to be an extension of the guide, the flare can be a separate piece that could be attached and detached from the guide based on the surgical need.

In still yet another embodiment of the preferred guide, the body of the guide is concave shaped. This enables the surgeon to make a curved incision on the proximal tibial surface.

In yet another embodiment of the preferred guide, the inner surface of the body exhibits a notch or a groove or a plurality of notches or grooves that provides a landmark or landmarks for rotation measurement, placement, or both of the tibia of an animal undergoing osteotomy.

In yet another embodiment of the preferred guide, the body evidences a plurality of orifices or fixation holes in the area enclosed by the first end, second end, inner surface and outer surface of the guide. These orifices or fixation holes are used to place, replace or both, a mechanism to secure the guide to an animal's bones. The mechanism comprises means of fixing or fixtures to fix the guide to the anatomical features of an animal's knee. These mechanisms include but are not limited to stays, pins (e.g., sta-fix pins), wires (e.g., k-wires), clamps (e.g., point to point clamps), or similar fixtures to affix the guide to bone, or any combination thereof. An example of the k-wire that could be used for securing the guide is the small sized k-wire (0.045 inches).

In still yet another embodiment of the preferred guide that is the present invention, the guide is reusable and durable. In another embodiment of the preferred guide, the guide may be made of materials comprising stainless steel, resin, titanium, tungsten, carbon steel, a combination thereof, or any material suitable for use in osteotomy.

In yet another embodiment the guide that is the present invention may be a single use device which is disposable.

In another preferred functional embodiment of the present invention Inventor envisions a method of performing a TPLO procedure using the disclosed device. Such a method comprises sedating the animal, shaving the area of skin over the tibia of the animal where the procedure will be performed; making an incision on the skin and using fingers or forceps to expose the muscle and bone underneath; protecting the patellar tendon of the animal; placing an osteotomy guide having a first end and second end on surface of the proximal tibia of the animal after measuring the absolute displacement of the tip of the osteotomy blade, said guide comprising a body with a tibial plateau facing inner surface, and an outer surface that is opposite to the inner surface, wherein the inner surface exhibits a notch or groove or a plurality of notches or grooves; securing the guide to the surface of the tibia after confirming that the notch or groove or notches or grooves are in proper orientation; using the osteotomy blade to make a curved incision along the tibial plateau facing inner surface of the body of the guide; marking the site for rotation of the tibia using the notch or groove or notches or grooves on the guide as a landmark or landmarks; rotating the tibia so that the tibia and femur are aligned properly; removing the guide from the proximal tibia of the animal; placing a metal bone plate to set the tibia in desired position; and securing the metal bone plate to the tibia and femur. The bone plate may be made of metal or any other material that is biocompatible.

In one embodiment of the preferred method, the body of the guide is concave shaped.

In another embodiment of the preferred method, the first end and second end of the body are flush or wherein the second end of the body is flush and said body has a flare protruding outward from the first end, said flare comprising a first end and a second end.

In yet another embodiment of the preferred method, the first end of the flare is an extension of the first end of the body and the second end of the flare is close to patellar tendon of the animal and protects the patellar tendon of the animal. Although the invention describes the flare to be an extension of the guide, the flare can be a separate piece that could be attached to and detached from the guide based on the surgical need.

In still yet another embodiment of the preferred method, the body has plurality of orifices or fixation holes in the area enclosed by the first end, second end, inner surface and outer surface of the guide. These orifices or fixation holes may be placed regularly or irregularly and be of the same or various shapes and sizes. These same orifices or fixation holes are used to place mechanisms to secure the guide to bone, where the mechanism includes but is not limited to pins (e.g., sta-fix pins), wires (e.g., k-wires), clamps (e.g., point to point clamps), any other mechanisms or fixtures for attachment and securement, or any combination thereof. An example of an exemplary wire, the k-wire, that could be used for securing the guide is the small sized k-wire (0.045 inches).

In another embodiment of the preferred method, the guide is reusable and durable.

In still another embodiment, the preferred method may incorporate a guide that is single use and disposable.

In yet another embodiment of the preferred method, the guide is made of materials including but not limited to stainless steel, resin, titanium, tungsten, carbon steel, a combination thereof, or any material suitable for use in osteotomy.

In yet another embodiment of the preferred method, the guides are of different sizes that are compatible with TPLO crescent blades and size of the tibia of an animal undergoing osteotomy.

In still yet another embodiment of the preferred method, the guide prevents/eliminates blade “drift”, movement and displacement and improves accuracy and efficiency of an osteotomy procedure.

### BRIEF DESCRIPTION OF THE DRAWINGS

So that the manner in which the present invention can be better understood, certain illustrations, charts and/or flow charts are appended hereto. It is to be noted, however, that the drawings illustrate only selected embodiments of the inventions and are therefore not to be considered limiting of scope, for the inventions can admit to other equally effective embodiments and applications.

Moreover, advantages and other aspects of the invention will be readily appreciated by those persons having skill in the art and may be better understood with further reference to the accompanying drawings in which like reference characters designate like or similar elements throughout the several figures of the drawings and wherein:

FIGS. 1A and 1B illustrate views of different versions of the osteotomy guide.

FIG. 1C illustrates the different sizes of the osteotomy guides that are compatible with TPLO blades.

FIG. 2 illustrates view of placement of the guide on the proximal tibial surface of an animal during TPLO procedure.

However, it should be understood that the above described figures are not intended to be limited to only the invention illustrated and to the particular embodiments disclosed, but on the contrary, the intention is intended to disclose all modifications, alternatives and equivalents falling within the spirit and scope of the invention as defined within the claim's broadest reasonable interpretation consistent with the specification. To wit, variations in height, length, width made be accomplished as to accommodate variances in sizes of animals being restrained, as may be necessary or required.

### DETAILED DESCRIPTION OF THE PREFERRED EMBODIMENTS

A detailed description of the preferred embodiments of the invention is disclosed and described below. Yet, each and every possible feature, within the limits of the specification, are not disclosed as various iterations are postulated to be in the purview and contemplation of those having skill in the art. It is therefore possible for those that have requisite skill in the art to make and practice the disclosed invention while observing that certain features and spatial arrangements are relative and capable of being scaled, adapted, arranged at various points about the present invention (by inventor, manufacturer or both) that nonetheless accomplishes the remediation of one or more of the infirmities as outlined and discussed above in the field of TPLO guide design and use. As well, individual components of the present invention may be sized (i.e. enlarged or shrunken) so long as the proportions are maintained that allow for maintaining of esthetic appearance and/or proper functioning of the present invention that may be sized and shaped according to the size of the animal undergoing the surgical procedure.

Equally, it should be observed that the present invention can be understood, in terms of structure, function, or both, from the present disclosure as well as those appended claims taken in context with the associated drawings. And whereas the present invention and method of use are capable of several different embodiments and permutations, which can be modified into several different configurations, each exhibiting accompanying interchangeable functionalities, without departing from the scope and spirit of the present application as shown and described.

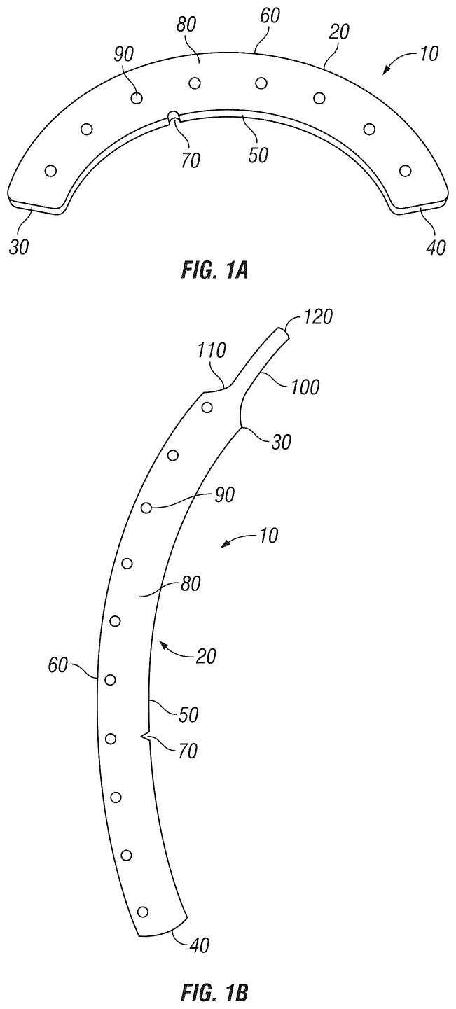

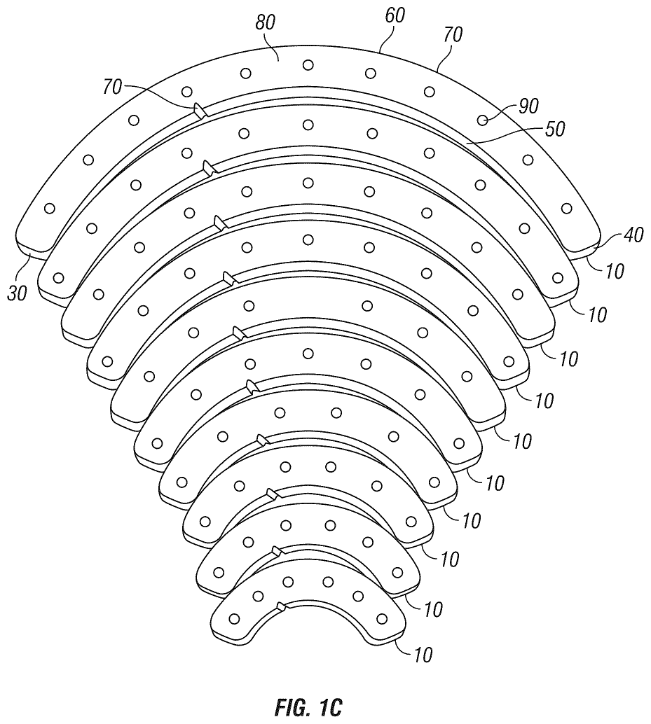

As detailed in FIG. 1A, one version of the osteotomy guide 10 comprises a concave shaped body 20 that is flushed on first end 30 and second end 40. The body 20 also comprises a tibial plateau facing inner surface 50 and an outer surface 60 that is opposite to the inner surface 50. The area 80 is enclosed by the inner surface 50, outer surface 60, first end 30, and second end 40. Present in area 80 are plurality of orifices or fixation holes 90a, 90b, 90c, . . . . (collectively referred to as orifice or fixation holes 90). There are various ways of arranging these orifices or fixation holes on area 80, which could be based on how a practitioner prefers to use the guide 10 and/or how hole 90 placement may enhance the surgical efficacy. One example is illustrated in FIG. 1A where the orifices or fixation holes are linearly arranged. Alternatively, holes 90 may be arranged near first end 30 and second end 40, placed sequentially, uniformly, randomly or some combination thereof. Moreover, the holes 90 themselves, although depicted as circular, may be of various shapes (i.e., circular, square, diamond, oval, rectangular, angular, curved, or a combination thereof). Too, holes 90 may have indentions, depressions, levels, notches, guides, gradation, or any combination thereof, for enhanced acceptance, guidance, or retention of pins, screws, wires, clamps, stays, rivets, or any combination thereof. The inner surface 50 exhibits a notch or groove 70 that can be used as a landmark for rotation of the tibia during osteotomy.

FIG. 1B illustrates a slightly modified version of guide 10. In this figure, body 20 comprises a flushed second end 40 and a flare 100 protruding out from first end 30. Flare 100 comprises first end 110 and second end 120, wherein the first end 110 is an extension of first end 30 and the second end 120 narrows to a flushed terminal end. Although flare 100 in FIG. 1B is an extension of the body 20, flare 100 could be a separate piece that can be attached at or detached from first end 30. Body 20 in FIG. 1B also comprises a tibial plateau facing inner surface 50 and an outer surface 60 that is opposite to inner surface 50. Area 80 is enclosed by inner surface 50, outer surface 60, second end 40, and second end 120. Present in area 80 are one to a plurality of orifices or fixation holes 90a, 90b, 90c, . . . . (collectively referred to as orifice or fixation holes 90). While depicted in series along in close proximity to outer surface 60, there are however various ways of arranging these orifices or fixation holes on area 80, which could be based on how the practitioner uses or intends to use the guide 10 and/or how it will enhance the surgical accuracy and efficacy. One example is illustrated in FIG. 1B where the orifices or fixation holes are linearly arranged. Alternatively, holes 90 may be arranged near first end 30 and/or second end 40, may be placed regularly or irregularly along and with area 80, and/or may be grouped or equidistantly placed. The inner surface 50 has a notch or groove 70 that can be used as a landmark for rotation of the tibia during osteotomy. Yet, Inventor envisions further one to a plurality of notches or grooves 70 that may be utilized to adjust, correct, place and replace the device that is the osteotomy guide 10.

As illustrated in FIG. 1C, osteotomy guide 10 is available in different sizes that are compatible with TPLO crescent blades, size and shape of the tibia of an animal undergoing osteotomy or a combination thereof. Guide 10 can be used for left or right stifle (i.e. knee joint). The components of guide 10 illustrated in FIG. 1C are the same as in FIG. 1A. These may also have flare 100 as described in FIG. 1B. The guides are disposable or reusable and durable being made of materials including but not limited to stainless steel, resin, titanium, tungsten, carbon steel, a combination thereof.

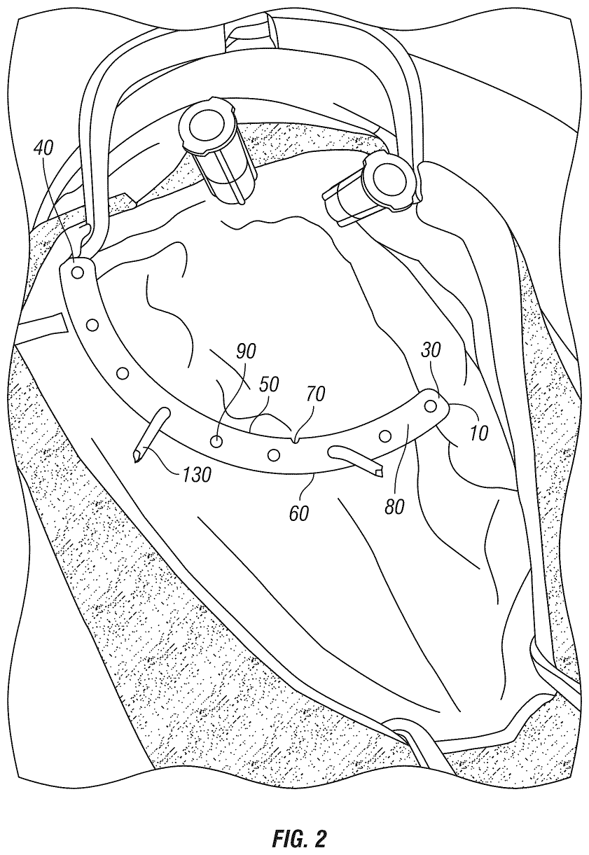

According to one method, after sedating the animal, the skin overlying the area on tibia of the animal where the TPLO procedure will be performed is shaved. An incision is made on the skin and the muscle and bone underneath are exposed using fingers or forceps. After making sure that the patellar tendon of the animal is protected, osteotomy guide 10 described in FIG. 1A and FIG. 1B is placed on surface of the proximal tibia of the animal after measuring the absolute displacement of the tip of the osteotomy guide (commonly known as “D” measurement). The placement of guide 10 is shown in FIG. 2, where the inner surface 50 faces the tibial plateau while the outer surface 60 is on the opposite side of inner surface 50. After confirming that the notch or groove 70, or notches and grooves 70, are in proper orientation, guide 10 is secured to the surface of tibia by mechanisms that include but are not limited to pins (e.g., sta-fix pin), wires (e.g., k-wire), clamps (e.g., point to point clamps) or any combination thereof.

As illustrated in FIG. 2, these securing mechanisms 130a, 130b, 130c, . . . (collectively referred to as securing mechanisms 130). The securing mechanisms 130 are placed or inserted in one or more of the orifices or fixation holes 90. Once guide 10 is secured, a curved incision is made along inner surface 50 using an osteotomy blade. Guide 10 prevents the “drifting”, movement or displacement of osteotomy blade 10 while making this incision, thereby increasing the efficiency and accuracy of the procedure. Additionally, notch or groove 70 is used as a landmark to mark the site for rotation of tibia, further increasing the efficiency and accuracy of the procedure. Once the rotation site is marked, the tibia is rotated so that it is aligned properly or correctly with the femur. Guide 10 is then removed from the proximal tibia of the animal and a metal bone plate is placed to set the tibia in relation to the femur and in the desired position, which is then secured.

Although only a select few example embodiments have been described in detail above, those skilled in the art will readily appreciate that many modifications are possible in the example embodiments without materially departing from this disclosure of the invention. Accordingly, all such modifications are intended to be included within the scope of this disclosure as defined in the appended claims.

What is claimed is:

1. An osteotomy guide, comprising:
a body with tibial plateau facing inner surface;
said inner surface along which an incision is made during osteotomy; and
an outer surface that is opposite to the inner surface, wherein said body has a first end and a second end;
wherein said guide prevents/eliminates blade drift and displacement and improves accuracy and efficiency of osteotomy procedure.

2. The osteotomy guide of claim 1, wherein the first end and second end of the body are flush or wherein the second end of the body is flush and said body has a flare protruding outward from the first end, said flare comprising a first end and a second end.

3. The osteotomy guide of claim 2, wherein the first end of the flare is an extension of the first end of the body and the second end of the flare is close to a patellar tendon of an animal undergoing osteotomy.

4. The osteotomy guide of claim 1, wherein the body of the guide is concave shaped.

5. The osteotomy guide of claim 1, wherein the inner surface of the body has a notch or a groove, or plurality of notches or groves, that provides a landmark or landmarks for rotation measurement, placement, or both of tibia of an animal undergoing osteotomy.

6. The osteotomy guide of claim 1, wherein the body has plurality of orifices or fixation holes in the area enclosed by the first end, second end, inner surface and outer surface of the guide, said orifices or fixation holes used to place, replace, or both, a mechanism to secure the guide;
said mechanism comprising a means of fixing or fixtures to fix said guide to anatomical features of an animal's knee.

7. The osteotomy guide of claim 6, wherein the mechanism comprises stays, pins (e.g., sta-fix pins), wires (e.g., k-wires), clamps (e.g., point to point clamps), or similar fixtures for affixing the guide to bone, or any combination thereof.

8. The osteotomy guide of claim 1, wherein said guide is reusable and durable.

9. The osteo guide of claim 1, wherein said guide is disposable.

10. The osteotomy guide of claim 1, wherein said guide is made of materials comprising stainless steel, resin, titanium, tungsten, carbon steel, plastic, rubber, or a combination thereof, or any material suitable for use in osteotomy.

11. The osteotomy guide of claim 1, wherein said guide is of different sizes that are compatible with TPLO crescent blades and size and shape of the tibia of an animal undergoing osteotomy.

12. A method of performing TPLO procedure on an animal, comprising:
sedating the animal, shaving the skin area above a tibia of the animal where the procedure is performed;
making an incision on said skin and using fingers or forceps to expose the muscle and bone underneath;
protecting the patellar tendon of the animal;
placing an osteotomy guide having a first end and second end on surface of the proximal tibia of the animal after measuring the absolute displacement of the tip of the osteotomy blade;
said guide comprising a body with tibial plateau facing inner surface, and an outer surface that is opposite to the inner surface, wherein the inner surface has a notch or groove or a plurality of notches or grooves;
securing the guide to the surface of the tibia after confirming that the notch, groove, notches or grooves are in proper orientation and placement;
using the osteotomy blade to make a curved incision along the tibial plateau facing inner surface of the body of the guide;
marking the site for rotation of the tibia using the notch or groove or notches or grooves on the guide as a landmark or landmarks;
rotating the tibia so that the tibia and femur are aligned properly;
removing the guide from the proximal tibia of the animal; and
placing a metal bone plate to set the tibia in desired position; and
securing the metal bone plate.

13. The method of claim 12, wherein the body of the guide is concave shaped.

14. The method of claim 12, wherein the first end and second end of the body are flush or wherein the second end of the body is flush and said body has a flare protruding outward from the first end, said flare comprising a first end and a second end.

15. The method of claim 14, wherein the first end of the flare is an extension of the first end of the body and the second end of the flare in close approximation to a patellar tendon of the animal and protects the patellar tendon of the animal during the TPLO procedure.

16. The method of claim 12, wherein the body has plurality of orifices or fixation holes in the area enclosed by the first end, second end, inner surface and outer surface of the guide, said orifices or fixation holes used to place, replace, or both, a mechanism to secure the guide;
said mechanism comprising a means of fixing or fixtures to fix said guide to anatomical features of an animal's knee.

17. The method of claim 16, wherein the mechanism comprises stays, pins (e.g., sta-fix pins), wires (e.g., k-wires), clamps (e.g., point to point clamps), or similar fixtures for affixing the guide to bone, or any combination thereof.

18. The method of claim 12, wherein said guide is reusable and durable.

19. The method of claim 12, wherein said guide is disposable.

20. The method of claim 12, wherein said guide is made of materials comprising stainless steel, resin, titanium, tungsten, carbon steel, plastic, rubber, a combination thereof, or any material suitable for use in osteotomy.

21. The method of claim 12, wherein the guides are of different sizes that are compatible with TPLO crescent blades and size and shape of the tibia of an animal undergoing osteotomy.

22. The method of claim 12, wherein said guide prevents/eliminates blade drift, displacement and improves accuracy and efficiency of osteotomy procedures.
